(19) United States
(12) Patent Application Publication
(10) Pub. No.: US 2021/0355216 A1
(43) Pub. Date: Nov. 18, 2021

(54) METHODS AND SYSTEMS FOR CONTROLLING THE AGONISTIC PROPERTIES OF ANTIBODY VARIABLE DOMAINS BY LIGHT

(71) Applicants: INSERM (INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); UNIVERSITÉ D'AIX MARSEILLE, Marseille Cedex 07 (FR)

(72) Inventors: Rémy LASSERRE, Marseille Cedex 09 (FR); Hai-Tao HE, Marseille Cedex 09 (FR); Amandine ANASTASIO, Lausanne Vaud (CH); Morgane JAEGER, Marseille Cedex 09 (FR)

(21) Appl. No.: 17/282,491

(22) PCT Filed: Oct. 4, 2019
(86) PCT No.: PCT/EP2019/076914

§ 371 Date: Apr. 2, 2021

(30) Foreign Application Priority Data
Oct. 5, 2018 (EP) 18306314.8

(51) Int. Cl. C07K 16/28; C07K 16/16; A61K 41/00

(52) U.S. Cl. CPC C07K 16/2809; C07K 16/16; A61K 41/00; C07K 2317/56; A61K 2039/505; C07K 2319/22; C07K 2317/75; C07K 2319/30; C07K 2317/55

(57) ABSTRACT

The inventors has developed a recombinant molecular system, named OptoFab, allowing the accurate control of the agonistic properties of an antibody-derived Fab fragment in time and in space using specific wavelengths of light. It consists in a Fab fragment derived from an agonistic antibody of interest, linked to optogenetic modules that confer a light response capacity. Indeed, antibody derived Fab fragments generally keep the specificity of the antibody for its epitope, but not its agonistic properties. However, when Fab fragments are oligomerized, they recover the agonistic properties of the whole antibody. These characteristics, are at the basis of the OptoFab concept as its objective is to manipulate the oligomerization/immobilization statue of a Fab fragment using optogenetics to control its agonistic property. The present invention relates to methods and systems for controlling the agonistic properties of antibody variable domains by light.

### FIELD OF THE INVENTION

The present invention relates to methods and systems for controlling the agonistic properties of antibody variable domains by light.

### BACKGROUND OF THE INVENTION

Studying the influence of the dynamics of signals perceived by cells on the quality of their response is becoming a new field of investigation in all areas of biomedical research. Indeed, thanks to the new technologies allowing studies of cellular responses at the level of the living single cell, it appears that in many cellular systems (neurons, fibroblasts, endothelial cells . . . ), the dynamics of the signals perceived by the cells (duration, frequency) strongly influences their functions or their fate. In order to progress in our understanding of the mechanisms by which this phenomenon is explained, the development of new tools allowing precise control of dynamic parameters of cellular stimulation in time and space is necessary.

### SUMMARY OF THE INVENTION

The present invention relates to methods and systems for controlling the agonistic properties of antibody variable domains by light. In particular, the present invention is defined by the claims.

### DETAILED DESCRIPTION OF THE INVENTION

The inventors has developed a recombinant molecular system, named OptoFab, allowing the accurate control of the agonistic properties of an antibody-derived Fab fragment in time and in space using specific wavelengths of light. It consists in a Fab fragment derived from an agonistic antibody of interest, linked to optogenetic modules that confer a light response capacity. Indeed, antibody derived Fab fragments generally keep the specificity of the antibody for its epitope, but not its agonistic properties. However, when Fab fragments are immobilized or oligomerized, they recover the agonistic properties of the whole antibody. These characteristics that are shared by numerous Fab fragments derived from agonistic antibodies, are at the basis of the OptoFab concept as its objective is to manipulate the oligomerization/immobilization statue of a Fab fragment using optogenetics to control its agonistic property.

Accordingly, the first object of the present invention relates to a recombinant protein comprising a variable domain of an antibody that is fused at its c-terminal end to a factor that can interact with a photoreceptor protein in a light-dependent manner.

As used herein, the term “variable domain” refers to both variable domains of immunoglobulin light chains and variable domains of heavy chain of an antibody.

As used herein the term “antibody” or “immunoglobulin” refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. The term also encompasses antibodies that are naturally devoid light chain that can be found e.g. in Camelid mammals. Thus the term encompasses single domain antibodies.

In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs.

As used herein, the term “single domain antibody” has its general meaning in the art and refers to the single heavy chain variable domain of antibodies of the type that can be found in Camelid mammals which are naturally devoid of light chains. Such single domain antibody are also called VHH or “Nanobody®”. For a general description of single domain antibodies, reference is made to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3

Thus, in some embodiments, the variable domain is selected from the group consisting of VH domains, VL domains, or single domain antibodies (sdAbs).

In some embodiments, the variable domain is a VH domain of a monoclonal antibody. As used herein, the term “monoclonal antibody” refer to a preparation of antibody molecules of single molecular composition. A monoclonal antibody composition displays a single binding specificity and affinity for a particular epitope.

In some embodiments, the recombinant protein of the present invention comprises a Fab fragment wherein the VH domain of the Fab fragment is fused at its c-terminal end to a factor that can interact with a photoreceptor protein in a light-dependent manner.

As used herein, the term “Fab fragment” has its general meaning in the art and refers to a monovalent fragment of an antibody consisting of the VL, VH, CL and CH1 domains. Fab fragments can be typically obtained, e.g., by treating an IgG antibody with papain. It is indeed well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). An antibody from which the Fc region has been enzymatically cleaved, or which has been produced without the Fc region, designated an Fab fragment, retains one of the antigen binding sites of an intact antibody molecule. Thus Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

In some embodiment, the Fab fragment derives from an antibody able to inhibit the function of its specific receptor.

In some embodiment, the Fab fragment derives from an antibody specific for a receptor and whose the monovalent form is not able to block the receptor function.

In some embodiments, the Fab fragment derives from an agonistic antibody.

In some embodiments, the Fab fragment derives from an agonistic antibody whose the monovalent form is not able to induce the biological signaling activity of the receptor.

As used herein, the term ‘agonistic antibody’ describes an antibody that is an agonist i.e. that is capable of stimulating the biological signalling activity of a receptor. As used herein, the term “receptor” has its general meaning in the art and denotes a cell-associated protein that binds to a bioactive molecule (i.e., a ligand) and mediates the effect of the ligand on the cell. Membrane-bound receptors are characterized by a multi-domain structure comprising an extracellular ligand-binding domain and an intracellular effector domain that is typically involved in signal transduction.

In some embodiments, the agonistic antibody is specific for a GPCR. As used herein, the term “G protein-coupled receptor” or “GPCR” has its general meaning in the art and refers to a large protein family of receptors, that sense molecules outside the cell and activate inside signal transduction pathways and, ultimately, cellular responses. The term is also known as seven-transmembrane domain receptors, 7TM receptors, heptahelical receptors, serpentine receptor, and G protein-linked receptors (GPLR). GPCRs can be grouped into 6 classes based on sequence homology and functional similarity: Class A (or 1) (Rhodopsin-like), Class B (or 2) (Secretin receptor family), Class C (or 3) (Metabotropic glutamate/pheromone), Class D (or 4) (Fungal mating pheromone receptors), Class E (or 5) (Cyclic AMP receptors) and Class F (or 6) (Frizzled/Smoothened). Examples of GPCRs include but are not limited to Chemokine (C-C motif) receptor 1 (CCR1, CKR1); Chemokine (C-C motif) receptor 2 (CCR2, CKR2); Chemokine (C-C motif) receptor 3 (CCR3, CKR3); Chemokine (C-C motif) receptor 4 (CCR4, CKR4); Chemokine (C-C motif) receptor 5 (CCR5, CKR5); Chemokine (C-C motif) receptor 8 (CCR8, CKR8); Chemokine (C-C motif) receptor-like 2 (CCRL2, CKRX); chemokine (C motif) receptor 1 (XCR1, CXC1) InterPro: IPR005393; chemokine (C-X3-C motif) receptor 1 (CX3CR1, C3X1) InterPro: IPR005387; GPR137B (GPR137B, TM7SF1); Chemokine receptor InterPro: IPR000355; Chemokine (C-C motif) receptor-like 1 (CCRL1 CCRL1, CCR11); Chemokine (C-C motif) receptor 6 (CCR6, CKR6); Chemokine (C-C motif) receptor 7 (CCR7, CKR7); Chemokine (C-C motif) receptor 9 (CCR9, CKR9); Chemokine (C-C motif) receptor 10 (CCR10, CKRA); CXC chemokine receptors InterPro: IPR001053; Chemokine (C-X-C motif) receptor 3 (CXCR3); Chemokine (C-X-C motif) receptor 4 (CXCR4, Fusin); Chemokine (C-X-C motif) receptor 5 (CXCR5); Chemokine (C-X-C motif) receptor 6 (CXCR6, BONZO); Chemokine (C-X-C motif) receptor 7 (CXCR7, RDC1) InterPro: IPR001416; Interleukin-8 InterPro: IPR000174 (IL8R); IL8R-α (IL8RA, CXCR1); IL8R-β(IL8RB, CXCR2); Adrenomedullin receptor (GPR182); Duffy blood group, chemokine receptor (DARC, DUFF); G Protein-coupled Receptor 30 (GPER, CML2, GPCR estrogen receptor); Angiotensin II receptor InterPro: IPR000248; Angiotensin II receptor, type 1 (AGTR1, AG2S); Angiotensin II receptor, type 2 (AGTR2, AG22); Apelin receptor (AGTRL1, APJ) InterPro: IPR003904; Bradykinin receptor InterPro: IPR000496; Bradykinin receptor B1 (BDKRB1, BRB1); Bradykinin receptor B2 (BDKRB2, BRB2); GPR15 (GPR15, GPRF); GPR25 (GPR25); Opioid receptor InterPro: IPR001418; delta Opioid receptor (OPRD1, OPRD); kappa Opioid receptor (OPRK1, OPRK); mu Opioid receptor (OPRM1, OPRM); Nociceptin receptor (OPRL1, OPRX); Somatostatin receptor InterPro: IPR000586; Somatostatin receptor 1 (SSTR1, SSR1); Somatostatin receptor 2 (SSTR2, SSR2); Somatostatin receptor 3 (SSTR3, SSR3); Somatostatin receptor 4 (SSTR4, SSR4); Somatostatin receptor 5 (SSTR5, SSR5); GPCR neuropeptide receptor InterPro: IPR009150; Neuropeptides B/W receptor 1 (NPBWR1, GPR7); Neuropeptides B/W receptor 2 (NPBWR2, GPR8); GPR1 orphan receptor (GPR1) InterPro: IPR002275; Galanin receptor InterPro: IPR000405; Galanin receptor 1 (GALR1, GALR); Galanin receptor 2 (GALR2, GALS); Galanin receptor 3 (GALR3, GALT); Cysteinyl leukotriene receptor InterPro: IPR004071; Cysteinyl leukotriene receptor 1 (CYSLTR1) Cysteinyl leukotriene receptor 2 (CYSLTR2) Leukotriene B4 receptor InterPro: IPR003981; Leukotriene B4 receptor (LTB4R, P2Y7); Leukotriene B4 receptor 2 (LTB4R2); Relaxin receptor InterPro: IPR008112; Relaxin/insulin-like family peptide receptor 1 (RXFP1, LGR7); Relaxin/insulin-like family peptide receptor 2 (RXFP2, GPR106); Relaxin/insulin-like family peptide receptor 3 (RXFP3, SALPR); Relaxin/insulin-like family peptide receptor 4 (RXFP4, GPR100/GPR142); KiSS1-derived peptide receptor (GPR54) (KISS1R) InterPro: IPR008103; Melanin-concentrating hormone receptor 1 (MCHR1, GPRO) InterPro: IPR008361; Urotensin-II receptor (UTS2R, UR2R) InterPro: IPR000670; Cholecystokinin receptor InterPro: IPR009126; Cholecystokinin A receptor (CCKAR, CCKR); Cholecystokinin B receptor (CCKBR, GASR); Neuropeptide FF receptor InterPro: IPR005395; Neuropeptide FF receptor 1 (NPFFR1, FF1R); Neuropeptide FF receptor 2 (NPFFR2, FF2R); Orexin receptor InterPro: IPR000204; Hypocretin (orexin) receptor 1 (HCRTR1, OX1R); Hypocretin (orexin) receptor 2 (HCRTR2, OX2R); Vasopressin receptor InterPro: IPR001817; Arginine vasopressin receptor 1A (AVPR1A, V1AR); Arginine vasopressin receptor 1B (AVPR1B, V1BR); Arginine vasopressin receptor 2 (AVPR2, V2R); Gonadotrophin releasing hormone receptor (GNRHR, GRHR) InterPro: IPR001658; Pyroglutamylated RFamide peptide receptor (QRFPR, GPR103); GPR22 (GPR22, GPRM); GPR176 (GPR176, GPR); Bombesin receptor InterPro: IPR001556; Bombesin-like receptor 3 (BRS3); Neuromedin B receptor (NMBR); Gastrin-releasing peptide receptor (GRPR); Endothelin receptor InterPro: IPR000499; Endothelin receptor type A (EDNRA, ET1R); Endothelin receptor type B (EDNRB, ETBR); GPR37 (GPR37, ETBR-LP2) InterPro: IPR003909; Neuromedin U receptor InterPro: IPR005390; Neuromedin U receptor 1 (NMUR1); Neuromedin U receptor 2 (NMUR2); Neurotensin receptor InterPro: IPR003984; Neurotensin receptor 1 (NTSR1, NTR1); Neurotensin receptor 2 (NTSR2, NTR2); Thyrotropin-releasing hormone receptor (TRHR, TRFR) InterPro: IPR009144; Growth hormone secretagogue receptor (GHSR) InterPro: IPR003905; GPR39 (GPR39); Motilin receptor (MLNR, GPR38); Anaphylatoxin receptors InterPro: IPR002234; C3a receptor (C3AR1, C3AR); C5a receptor (C5AR1, C5AR); Chemokine-like receptor 1 (CMKLR1, CML1) InterPro: IPR002258; Formyl peptide receptor InterPro: IPR000826; Formyl peptide receptor 1 (FPR1, FMLR); Formyl peptide receptor-like 1 (FPRL1, FML2); Formyl peptide receptor-like 2 (FPRL2, FML1); MAS1 oncogene InterPro: IPR000820; MAS1 (MAS1, MAS); MAS1L (MAS1L, MRG); GPR1 (GPR1); GPR32 (GPR32, GPRW); GPR44 (GPR44); GPR77 (GPR77, C5L2); Melatonin receptor InterPro: IPR000025; Melatonin receptor 1A (MTNR1A, ML1A); Melatonin receptor 1B (MTNR1B, ML1B); Neurokinin receptor InterPro: IPR001681; Tachykinin receptor 1 (TACR1, NK1R); Tachykinin receptor 2 (TACR2, NK2R); Tachykinin receptor 3 (TACR3, NK3R); Neuropeptide Y receptor InterPro: IPR000611; Neuropeptide Y receptor Y1 (NPY1R, NY1R); Neuropeptide Y receptor Y2 (NPY2R, NY2R); Pancreatic polypeptide receptor 1 (PPYR1, NY4R); Neuropeptide Y receptor Y5 (NPY5R, NY5R); Prolactin-releasing peptide receptor (PRLHR, GPRA) InterPro: IPR001402; Prokineticin receptor 1 (PROKR1, GPR73); Prokineticin receptor 2 (PROKR2, PKR2); GPR19 (GPR19, GPRJ); GPR50 (GPR50, ML1X); GPR75 (GPR75); GPR83 (GPR83, GPR72); Glycoprotein hormone receptor InterPro: IPR002131; FSH-receptor (FSHR); Luteinizing hormone/choriogonadotropin receptor (LHCGR, LSHR); Thyrotropin receptor (TSHR); Leucine-rich repeat-containing G protein-coupled receptor 4 (LGR4, GPR48); Leucine-rich repeat-containing G protein-coupled receptor 5 (LGR5, GPR49); Leucine-rich repeat-containing G protein-coupled receptor 6 (LGR6); GPR40-related receptor InterPro: IPR013312; Free fatty acid receptor 1 (FFAR1, GPR40); Free fatty acid receptor 2 (FFAR2, GPR43); Free fatty acid receptor 3 (FFAR3, GPR41); GPR42 (GPR42, FFAR1L); P2 purinoceptor InterPro: IPR002286; Purinergic receptor P2Y1 (P2RY1); Purinergic receptor P2Y2 (P2RY2); Purinergic receptor P2Y4 (P2RY4); Purinergic receptor P2Y6 (P2RY6); Purinergic receptor P2Y8 (P2RY8); Purinergic receptor P2Y11 (P2RY11); Hydroxycarboxylic acid receptor 1 (HCAR1, GPR81); Hydroxycarboxylic acid receptor 2, Niacin receptor 1 (HCAR2, GPR109A); Hydroxycarboxylic acid receptor 3, Niacin receptor 2 (HCAR3, GPR109B, HM74); GPR31 (GPR31, GPRV); GPR82 (GPR82); Oxoglutarate (alpha-ketoglutarate) receptor 1 (OXGR1, GPR80); Succinate receptor 1 (SUCNR1, GPR91); P2 purinoceptor InterPro: IPR002286; Purinergic receptor P2Y12 (P2RY12); Purinergic receptor P2Y13 (P2RY13, GPR86) InterPro: IPR008109; Purinergic receptor P2Y14 (P2RY14, UDP-glucose receptor, KI01) InterPro: IPR005466; GPR34 (GPR34); GPR87 (GPR87); GPR171 (GPR171, H963); Platelet-activating factor receptor (PTAFR, PAFR) InterPro: IPR002282; Cannabinoid receptor InterPro: IPR002230; Cannabinoid receptor 1 (brain) (CNR1, CB1R); Cannabinoid receptor 2 (macrophage) (CNR2, CB2R); Lysophosphatidic acid receptor InterPro: IPR004065; Lysophosphatidic acid receptor 1 (LPAR1); Lysophosphatidic acid receptor 2 (LPAR2); Lysophosphatidic acid receptor 3 (LPAR3); Sphingosine 1-phosphate receptor InterPro: IPR004061; Sphingosine 1-phosphate receptor 1 (S1PR1); Sphingosine 1-phosphate receptor 2 (S1PR2); Sphingosine 1-phosphate receptor 3 (S1PR3); Sphingosine 1-phosphate receptor 4 (S1PR4); Sphingosine 1-phosphate receptor 5 (S1PR5); Melanocortin/ACTH receptor InterPro: IPR001671; Melanocortin 1 receptor (MC1R, MSHR); Melanocortin 3 receptor (MC3R); Melanocortin 4 receptor (MC4R); Melanocortin 5 receptor (MC5R); ACTH receptor (MC2R), ACTR); GPR3 (GPR3); GPR6 (GPR6); GPR12 (GPR12, GPRC); Eicosanoid receptor InterPro: IPR008365; Prostaglandin D2 receptor (PTGDR, PD2R); Prostaglandin E1 receptor (PTGER1, PE21); Prostaglandin E2 receptor (PTGER2, PE22): Prostaglandin E3 receptor (PTGER3, PE23); Prostaglandin E4 receptor (PTGER4, PE24); Prostaglandin F receptor (TGFR, PF2R); Prostaglandin I2 (prostacyclin) receptor (PTGIR, PI2R); Thromboxane A2 receptor (TBXA2R, TA2R); Lysophosphatidic acid receptor InterPro: IPR004065; Lysophosphatidic acid receptor 4 (LPAR4); Lysophosphatidic acid receptor 5 (LPAR5); Lysophosphatidic acid receptor 6 (LPAR6) InterPro: IPR002188; P2 purinoceptor InterPro: IPR002286; Purinergic receptor P2Y10 (P2RY10, P2Y10), Protease-activated receptor InterPro: IPR003912; Coagulation factor II (thrombin) receptor-like 1 (F2RL1, PAR2); Coagulation factor II (thrombin) receptor-like 2 (F2RL2, PAR3); Coagulation factor II (thrombin) receptor-like 3 (F2RL3, PAR4); Epstein-Barr virus induced gene 2 (lymphocyte-specific G protein-coupled receptor) (GPR183); Proton-sensing G protein-coupled receptors; GPR4 (GPR4) InterPro: IPR002276; GPR65 (GPR65) InterPro: IPR005464; GPR68 (GPR68) InterPro: IPR005389; GPR132 (GPR132, G2A) InterPro: IPR005388; GPR17 (GPR17, GPRH); GPR18 (GPR18, GPRI); GPR20 (GPR20, GPRK); GPR35 (GPR35); PR55 (GPR55); Coagulation factor II receptor (F2R, THRR); Opsins InterPro: IPR001760; Rhodopsin (RHO, OPSD); Opsin 1 (cone pigments), short-wave-sensitive (color blindness, tritan) (OPN1SW, OPSB) (blue-sensitive opsin); Opsin 1 (cone pigments), medium-wave-sensitive (color blindness, deutan) (OPN1MW, OPSG) (green-sensitive opsin); Opsin 1 (cone pigments), long-wave-sensitive (color blindness, protan) (OPN1LW, OPSR) (red-sensitive opsin); Opsin 3, Panopsin (OPN3); Opsin 4, Melanopsin (OPN4); Opsin 5 (OPN5, GPR136); Retinal G protein coupled receptor (RGR); Retinal pigment epithelium-derived rhodopsin homolog (RRH, OPSX) (visual pigment-like receptor opsin) InterPro: IPR001793; 5 Hydroxytryptamine (5-HT) receptor InterPro: IPR002231; 5-HT2A (HTR2A, 5H2A); 5-HT2B (HTR2B, 5H2B); 5-HT2C (HTR2C, 5H2C); 5-HT6 (HTR6, 5H6) InterPro: IPR002232; Adrenergic receptor InterPro: IPR002233; Alpha1A (ADRA1A, A1AA); Alpha1B (ADRA1B, A1AB); Alpha1D (ADRA1D, A1AD); Alpha2A (ADRA2A, A2AA); Alpha2B (ADRA2B, A2AB); Alpha2C (ADRA2C, A2AC); Beta1 (ADRB1, B1AR); Beta2 (ADRB2, B2AR); Beta3 (ADRB3, B3AR); Dopamine receptor InterPro: IPR000929; D1 (DRD1, DADR); D2 (DRD2, D2DR); D3 (DRD3, D3DR); D4 (DRD4, D4DR4); D5 (DRD5, DBDR); Trace amine receptor InterPro: IPR009132; TAAR1 (TAAR1, TART); TAAR2 (TAAR2, GPR58); TAAR3 (TAAR3, GPR57); TAAR5 (TAAR5, PNR); TAAR6 (TAAR6, TAR4); TAAR8 (TAAR8, GPR102); TAAR9 (TAAR9, TAR3); Histamine H2 receptor (HRH2, HH2R) InterPro: IPR000503; Histamine H1 receptor (HRH1, HH1R) InterPro: IPR000921; Histamine H3 receptor (HRH3) InterPro: IPR003980; Histamine H4 receptor (HRH4) InterPro: IPR008102; Adenosine receptor InterPro: IPR001634; A1 (ADORA1, AA1R); A2a (ADORA2A, AA2A); A2b (ADORA2B, AA2B); A3 (ADORA3, AA3R); Muscarinic acetylcholine receptor InterPro: IPR000995; M1 (CHRM1, ACM1); M2 (CHRM2, ACM2); M3 (CHRM3, ACM3); M4 (CHRM4, ACM4); M5 (CHRM5, ACM5); GPR21 (GPR21, GPRL); GPR27 (GPR27); GPR45 (GPR45, PSP24); GPR52 (GPR52); GPR61 (GPR61); GPR62 (GPR62); GPR63 (GPR63); GPR78 (GPR78); GPR84 (GPR84); GPR85 (GPR85); GPR88 (GPR88); GPR101 (GPR101); GPR161 (GPR161, RE2); GPR173 (GPR173, SREB3); 5-Hydroxytryptamine (5-HT) receptor InterPro: IPR002231; 5-HT1A (HTR1A, SHIA); 5-HT1B (HTR1B, 5H1B); 5-HT1D (HTR1D, 5H1D); 5-HT1E (HTR1E, 5H1E); 5-HT1F (HTR1F, 5H1F); 5-HT4 (HTR4) InterPro: IPR001520; 5-HT5A (HTR5A, 5H5A); and 5-HT7 (HTR7, 5H7) InterPro: IPR001069.

In some embodiments, the agonistic antibody is specific for a tyrosine kinase receptor, in particular a RTK. As used herein the term “RTK” refers to the cell surface form of protein tyrosine kinase (E.C. 2.7.1.112) which cellular surface expression/activation is typically associated with the onset or progression of a disease, usually a malignant disease, such as cancer. The RTKs have been divided into a number of classes as follows: RTK class I (EGF receptor family); II (insulin receptor family); III (PDGR receptor family); IV (FGF receptor family); V (VEGF receptor family); VI (HGF receptor family); VII (Trk receptor family); VIII (AXL receptor family); IX (AXL receptor family); X (LTK receptor family); XI (TIE receptor family); XII (ROR receptor family); XIII (DDR receptor family); XV (KLG receptor family); XVI (RYK receptor family); arid XVII (MuSK receptor family). The RTKs that depend upon cytosolic receptors include T and B-cell receptors, integrins, interferon receptors, interleukin receptors, GP130 associated proteins, etc. Examples of RTKs include but are not mimited to EPOR, GHR, CFSR, PRLR, MPL; IFN Family: IFNAR1, 2, IFNGR1, 2; γC Family: IL2RA, B, G, IL4R, IL2RG (Type 1 receptor), IL4R-IL13RA1 (Type II receptor), IL7R, IL2RG, IL9R, IL15RA, IL2RB, IL10RA, B, IL12RB1, 2, IL13RA1; IL3 Family: IL3RA, CSF2RA, B, IL5RA, GP130 Family: IL6R, IL6ST, IL11RA, LIFR, OSMR, IL6GT, CNTFR, IL6ST, and LIFR. Examples of such receptors includes epidermal growth factor receptor (EGFR), insulin receptor, platelet-derived growth factor receptor (PDGFR), vascular endothelial growth factor receptor (VEGFR), fibroblast growth factor receptor (FGFR), hepatocyte growth factor receptor (HGFR), and nerve growth factor receptor (NGFR). In some embodiments, the RTK is a member of the EGFR family such as EGFR or erbB-1, erbB-2, erbB-3, or erbB-4. More preferably, the RTK is EGFR, which is a 170 kDa membrane-spanning glycoprotein that binds to, for example, EGF, TNF-α, amphiregulin, heparin-binding EGF (HB-EGF), betacellulin, epiregulin, and NRG2-α. Also preferably, the RTK is HER2, a proto-oncogene that encodes a transmembrane receptor protein of 185 kDa. The RTK may also be a member of the VEGF receptor (VEGFR) family, which includes VEGFR-1, VEGFR-2, VEGFR-3, neuropilin-1 and neuropilin-2. Ligands that bind to VEGFR-1 and VEGFR-2 include isoforms of VEGF (VEGF121, VEGF145, VEGF165, VEGF189 and VEGF206). In some embodiments, the RTK is a member oft the type III family of receptor tyrosine kinase. As used herein the term “type III family of receptor tyrosine kinases” or “type III RTKs” is intended to include receptor tyrosine kinases which typically contain five immunoglobulin like domains, or Ig-like domains, in their ectodomains. Examples of type III RTKs include, but are not limited to PDGF receptors, the M-CSF receptor, the FGF receptor, the Flt3-receptor (also known as Flk2) and the KIT receptor. In a preferred embodiment of the invention, the type III RTK is KIT (also known in the art as the SCF receptor). KIT, like other type III RTKs is composed of a glycosylated extracellular ligand binding domain (ectodomain) that is connected to a cytoplasmic region by means of a single transmembrane (TM) domain (reviewed in Schlessinger (2000) Cell 103: 211-225). Another hallmark of the type III RTKs, e.g., KIT, is a cytoplasmic protein tyrosine kinase (PTK) domain with a large kinase-insert region. At least two splice iso forms of the KIT receptor are known to exist, the shorter making use of an in-frame splice site. All iso forms of KIT, and the other above described RTKs, are encompassed by the present invention. The terms “Kit”, “KIT” and “KIT receptor”, as used herein, include the type III transmembrane receptor tryosine kinase (RTK) that plays crucial roles in mediating diverse cellular processes including cell differentiation, proliferation and cell survival, among other activities, upon binding by any KIT ligand, e.g., Stem Cell Factor (SCF) (reviewed in Schlessinger (2000) Cell 103: 211-225). KIT is also known as the SCF receptor. Like other members of the type III subfamily of RTKs, KIT is composed of an extracellular domain that includes five Ig-like domains (designated D1-D5), a single transmembrane domain, a juxtamembrane region, a tyrosine kinase domain split by a kinase insert and a C-terminal tail. In some embodiments of the invention, the KIT is human KIT. The term “KIT” is also intended to include recombinant human KIT (rh KIT), which can be prepared by standard recombinant expression methods.

In some embodiments, the agonistic antibody is specific for a receptor of an immune cell. In particular, the antibody may be specific for an immune cell regulatory molecule such as CD3, CD4, CD8, CD25, CD28, CD26, CTLA-4, ICOS, or CD11a. Other suitable antigens include but are not limited to those associated with immune cells including T cell-associated molecules, such as TCR/CD3 or CD2; NK cell-associated targets such as NKG2D, FcγRIIIa (CD16), CD38, CD44, CD56, or CD69; granulocyte-associated targets such as FcγRI (CD64), FcαRI (CD89), and CR3 (CD11b/CD18); monocyte/macrophage-associated targets (such as FcγRI (CD64), FcαRI (CD89), CD3 (CD11b/CD18), or mannose receptor; dendritic cell-associated targets such as FcγRI (CD64) or mannose receptor; and erythrocyte-associated targets such as CRI (CD35).

In some embodiments, the agonistic antibody is specific for a TCR. As used herein, the term “TCR” has its general meaning in the art and refers to the molecule found on the surface of T cells that is responsible for recognizing antigens bound to MHC molecules. During antigen processing, antigens are degraded inside cells and then carried to the cell surface in the form of peptides bound to major histocompatibility complex (MHC) molecules (human leukocyte antigen HLA molecules in humans). T cells are able to recognize these peptide-MHC complex at the surface of professional antigen presenting cells or target tissue cells such as β cells in T1D. There are two different classes of MHC molecules: MHC Class I and MHC Class II that deliver peptides from different cellular compartments to the cell surface that are recognized by CD8+ and CD4+ T cells, respectively. The T cell receptor or TCR is the molecule found on the surface of T cells that is responsible for recognizing antigens bound to MHC molecules. The TCR heterodimer consists of an alpha and beta chain in 95% of T cells, whereas 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. Each chain of the TCR is a member of the immunoglobulin superfamily and possesses one N-terminal immunoglobulin (Ig)-variable (V) domain, one Ig-constant (C) domain, a transmembrane region, and a short cytoplasmic tail at the C-terminal end. The constant domain of the TCR consists of short connecting sequences in which a cysteine residue forms a disulfide bond, making a link between the two chains. The structure allows the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The CD3 chains, together with the TCR, form what is known as the TCR complex. The signal from the TCR complex is enhanced by simultaneous binding of the MHC molecules by a specific co-receptor. On helper T cells, this co-receptor is CD4 (specific for class II MHC); whereas on cytotoxic T cells, this co-receptor is CD8 (specific for class I MHC). The co-receptor not only ensures the specificity of the TCR for an antigen, but also allows prolonged engagement between the antigen presenting cell and the T cell and recruits essential molecules (e.g., LCK) inside the cell involved in the signaling of the activated T lymphocyte. The term “T-cell receptor” is thus used in the conventional sense to mean a molecule capable of recognising a peptide when presented by an MHC molecule. The molecule may be a heterodimer of two chains α and β (or optionally γ and δ) or it may be a recombinant single chain TCR construct. The variable domain of both the TCR α-chain and β-chain have three hypervariable or complementarity determining regions (CDRs). CDR3 is the main CDR responsible for recognizing processed antigen. Its hypervariability is determined by recombination events that bring together segments from different gene loci carrying several possible alleles. The genes involved are V and J for the TCR α-chain and V, D and J for the TCR β-chain. Further amplifying the diversity of this CDR3 domain, random nucleotide deletions and additions during recombination take place at the junction of V-J for TCR α-chain, thus giving rise to V(N)J sequences; and V-D and D-J for TCR β-chain, thus giving rise to V(N)D(N)J sequences. Thus, the number of possible CDR3 sequences generated is immense and accounts for the wide capability of the whole TCR repertoire to recognize a number of disparate antigens. At the same time, this CDR3 sequence constitutes a specific molecular fingerprint for its corresponding T cell. The CDR3 amino acid and nucleotide sequences of the TCR characterized by the inventors are listed in the following Table A. Rearranged nucleotide sequences are presented as V segments (underlined) followed by (ND)N segments (not underlined; N additions denoted in bold) and then by J segments (underlined), as annotated using the IMGT database (www.imgt.org).

In some embodiments, the agonistic antibody is specific for a costimulatory receptor. As used herein, the term “costimulatory receptor” includes receptors which transmit a costimulatory signal to an immune cell. In some embodiments, costimulatory receptor is selected from the group consisting of CD134 (OX40), CD137 (4-1BB), CD28, GITR, CD27, CD70, ICOS, RANKL, TNFRSF25 (DR3), CD258 (LIGHT), CD40, HVEM, and the like.

In some embodiments, the agonistic antibody is specific for a receptor selected from the group consisting of CD1a, CD1b, CD1c, CD1d, CD1e, CD2, CD3delta, CD3epsilon, CD3gamma, CD4, CD5, CD6, CD7, CD8alpha, CD8beta, CD9, CD10, CD11a, CD11b, CD11c, CDw12, CD13, CD14, CD15u, CD16a, CD16b, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD44R, CD45, CD46, CD47R, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CD75, CD75s, CD77, CD79a, CD79b, CD80, CD81, CD82, CD83, CD84, CD85, CD86, CD87, CD88, CD89, CD90, CD91, CD92, CDw93, CD94, CD95, CD96, CD97, CD98, CD99, CD100, CD101, CD102, CD103, CD104, CD105, CD106, CD107a, CD107b, CD108, CD109, CD110, CD111, CD112, CDw113, CD114, CD115, CD116, CD117, CD118, CDw119, CD120a, CD120b, CD121a, CDw121b, CD122, CD123, CD124, CDw125, CD126, CD127, CDw128a, CDw128b, CD129, CD130, CD131, CD132, CD133, CD134, CD135, CDw136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156a, CD156b, CDw156C, CD157, CD158, CD159a, CD159c, CD160, CD161, CD162, CD162R, CD163, CD164, CD165, CD166, CD167a, CD168, CD169, CD170, CD171, CD172a, CD172b, CD172g, CD173, CD174, CD175, CD175 s, CD176, CD177, CD178, CD179a, CD179b, CD180, CD181, CD182, CD183, CD184, CD185, CDw186, CD191, CD192, CD193, CD195, CD196, CD197, CDw198, CDw199, CDw197, CD200, CD201, CD202b, CD203c, CD204, CD205, CD206, CD207, CD208, CD209, CDw210, CD212, CD213a1, CD213a2, CDw217, CDw218a, CDw218b, CD220, CD221, CD222, CD223, CD224, CD225, CD226, CD227, CD228, CD229, CD230, CD231, CD232, CD233, CD234, CD235a, CD235b, CD235ab, CD236, CD236R, CD238, CD239, CD240CE, CD240D, CD240DCE, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD271, CD272, CD273, CD274, CD275, CD276, CD277, CD278, CD279, CD280, CD281, CD282, CD283, CD284, CD289, CD292, CDw293, CD294, CD295, CD296, CD297, CD298, CD299, CD300a, CD300c, CD300e, CD301, CD302, CD303, CD304, CD305, CD306, CD307, CD309, CD312, CD314, CD315, CD316, CD317, CD318, CD319, CD320, CD321, CD322, CD324, CDw325, CD326, CDw327, CDw328, CDw329, CD331, CD332, CD333, CD334, CD335, CD336, CD337, CDw338 and CD339.

Methods for producing antibodies are well known in the art. For instance, monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with the appropriate antigenic forms (i.e. receptor of interest). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant receptor of interest may be provided by expression with recombinant cell lines. Recombinant forms of the polypeptides may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods. Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Many agonistic antibodies are known in the art. For instance, anti-OX40 antibodies are described, for example, in U.S. Pat. Nos. 8,614,295; 7,501,496; and 8,283,450, incorporated herein by reference in their entirety for the disclosure of anti-OX40 antibodies. Anti-4-1BB antibodies are described, for example, in U.S. Pat. Nos. 6,569,997; 6,974,863; and 8,137,667, incorporated herein by reference in their entirety for the disclosure of anti-4-1BB antibodies. Anti-CD28 antibodies are described, for example, in U.S. Pat. Nos. 7,585,960; 8,334,102, and 7,723,482, incorporated herein by reference in their entirety for the disclosure of anti-CD28 antibodies. Anti-GITR antibodies are described, for example, in U.S. Pat. Nos. 7,812,135 and 8,388,967, incorporated herein by reference in their entirety for the disclosure of anti-GITR antibodies. Anti-CD27 antibodies are described, for example, in U.S. Pat. No. 8,481,029, incorporated herein by reference in its entirety for the disclosure of anti-CD28 antibodies. Anti-CD70 antibodies are described, for example, in U.S. Pat. Nos. 8,337,838; 8,124,738; and 7,491,390, incorporated herein by reference in their entirety for the disclosure of anti-CD70 antibodies. Anti-ICOS antibodies are described, for example, in U.S. Pat. Nos. 7,521,532 and 8,318,905, incorporated herein by reference in their entirety for the disclosure of anti-ICOS antibodies. Anti-RANKL antibodies are described, for example, in U.S. Pat. Nos. 7,411,050; 8,414,890, and 8,377,690, incorporated herein by reference in their entirety for the disclosure of anti-RANKL antibodies. An exemplary anti-RANKL antibody is denosumab. Anti-TNFRSF25 (DR3) antibodies are described, for example, in U.S. Patent Publication Nos. US20130330360, and US20120014950 incorporated herein by reference in their entirety for the disclosure of anti-DR3 antibodies. Anti-CD258 (LIGHT) antibodies are described, for example, in U.S. Patent Publication Nos. US20130315913 and US20090214519, incorporated herein by reference in their entirety for the disclosure of anti-LIGHT antibodies. Anti-CD40 antibodies are described, for example, in U.S. Pat. Nos. 8,669,352; 8,637,032; 8,591,900; 8,492,531; 8,388,971; 8,303,955; 7,790,166; 7,666,422; 7,563,442; 7,537,763; and 7,445,780, incorporated herein by reference in their entirety for the disclosure of anti-CD40 antibodies. Anti-HVEM antibodies are described, for example, in U.S. Pat. Nos. 6,573,058, and 8,440,185, incorporated herein by reference in their entirety for the disclosure of anti-HVEM antibodies.

In some embodiments, factors that interact with a photoreceptor protein in a light-dependent manner are not particularly limited and include any proteins or protein fragments that are capable of binding to a cognate photoreceptor protein in a light-dependent manner, i.e. that bind to a photo-activated from of the photoreceptor, but not to a photo-inactivated from. In some embodiments, said factor is selected from the group consisting of Phytochrome Interacting Factors (PIFs), FHY1/FHL, Phytochrome kinase substrate 1 (PKS1), nucleoside diphosphate kinase 2 (NDPK2), cryptochromes such as CRY1 and CRY2, Aux/IAA proteins, phosphatases such as FyPP and PAPP5, E3 ubiquitin ligases such as COP1, and ARR4. Preferably, said factor is selected from the group consisting of PIF1, PIF2, PIF3, PIF4, PIF5, PIF6, and PIF7, wherein PIF6 is particularly preferred. Even more preferably, said factor consists of the first 100 amino-terminal amino acids of PIF6. In one particular example, said factor can be PIF6 derived from Arabidopsis thaliana. In some embodiments, the factor comprises an amino acid sequence that has at least 90% of identity with the amino acid sequence as set forth in SEQ ID NO: 1.

SEQ ID NO: 1
MFLPTDYCCRLSDQEYMELVFENGQILAKGQRSNVSLHNQRTKSIMDLYEA
EYNEDFMKSIIHGGGGAITNLGDTQVVPQSHVAAAHETNMLESNKHVDGSG
SGSGSGSENLYFQG

According to the invention a first amino acid sequence having at least 90% of identity with a second amino acid sequence means that the first sequence has 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155 165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

In some embodiments, the variable domain and the factor are fused to each other directly (i.e. without use of a linker) or via a linker. The linker is typically a linker peptide and will, according to the invention, be selected so as to allow binding of the polypeptide to the heterologous polypeptide. Suitable linkers will be clear to the skilled person based on the disclosure herein, optionally after some limited degree of routine experimentation. Suitable linkers are described herein and may—for example and without limitation—comprise an amino acid sequence, which amino acid sequence preferably has a length of 2 or more amino acids. Typically, the linker has 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. The linker sequence may be a naturally occurring sequence or a non-naturally occurring sequence. One useful group of linker sequences are linkers derived from the hinge region of heavy chain antibodies as described in WO 96/34103 and WO 94/04678. Other examples are poly-alanine linker sequences such as Ala-Ala-Ala. Further preferred examples of linker sequences are Gly/Ser linkers of different length including (gly4ser)3, (gly4ser)4, (gly4ser), (gly3ser), gly3, and (gly3ser2)3.

The recombinant protein of the present invention may be produced by any suitable means, as will be apparent to those of skill in the art. In order to produce sufficient amounts of polypeptides or functional equivalents thereof for use in accordance with the present invention, expression may conveniently be achieved by culturing under appropriate conditions recombinant host cells containing the polypeptide of the present invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells, HeLa cells, baby hamster kidney cells and many others. Bacteria are also preferred hosts for the production of recombinant protein, due to the ease with which bacteria may be manipulated and grown. A common, preferred bacterial host is E coli. Alternatively, the polypeptide of the present invention is produced by any technique known in the art, such as, without limitation, any chemical, biological, genetic or enzymatic technique, either alone or in combination. For example, knowing the amino acid sequence of the desired sequence, one skilled in the art can readily produce said polypeptide, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

A further aspect of the present invention relates to a nucleic acid encoding for the recombinant protein of the present invention. As used herein, the term “nucleic acid molecule” has its general meaning in the art and refers to a DNA or RNA molecule. However, the term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fiuorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylino sine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine. In some embodiments, the nucleic acid molecule of the present invention is included in a suitable vector, such as a plasmid, cosmid, episome, artificial chromosome, phage or a viral vector. So, a further object of the invention relates to a vector comprising a nucleic acid encoding for the Recombinant protein of the present invention. Typically, the vector is a viral vector which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector.

A further object of the present invention relates to a host cell transformed with the nucleic acid molecule of the present invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”. For instance, as disclosed above, for expressing and producing the polypeptide of the present invention, prokaryotic cells and, in particular E. coli cells, will be chosen. Actually, according to the invention, it is not mandatory to produce the Recombinant protein of the present invention in a eukaryotic context that will favour post-translational modifications (e.g. glycosylation). Typically, the host cell may be suitable for producing the polypeptide of the present invention as described above. In some embodiments, the host cells is isolated from a mammalian subject who is selected from a group consisting of: a human, a horse, a dog, a cat, a mouse, a rat, a cow and a sheep. In some embodiments, the host cell is a human cell. In some embodiments, the host cell is a cell in culture. The cells may be obtained directly from a mammal (preferably human), or from a commercial source, or from tissue, or in the form for instance of cultured cells, prepared on site or purchased from a commercial cell source and the like. In some embodiments, the host cell is a mammalian cell line (e.g., Vero cells, CHO cells, 3T3 cells, COS cells, etc.).

A further object of the present invention relates to an optogenetic system comprising at least one recombinant protein of the present invention and at least one photoreceptor protein.

Photoreceptor proteins to be used in the optogenetic system of the present invention are not particularly limited and include any protein or protein fragment that is capable of undergoing a conformational change in response to absorption of photons of a particular wavelength, and, as a consequence, displays binding to a particular binding partner in a light-dependent manner.

In some embodiments, the photoreceptor protein is a phytochrome. As used herein, the term “phytochrome” has its general meaning in the art and refers to a family of photosensory molecules that plants and bacteria use to monitor informational light signals in the environment. These molecules, together with other informational photoreceptors, including the cryptochromes and phototropins provide plants and bacteria with the capacity to continuously track the presence, absence, spectral quality, fluence rate, directionality and diurnal duration of incoming light signals, and to adjust their growth and development toward optimal radiant energy capture, survival and reproduction. In some embodiments, the phytochrome is selected from the group consisting of Phytochrome A (PhyA), Phytochrome B (PhyB), Phytochrome C (PhyC), Phytochrome D (PhyD), and Phytochrome E (PhyE).

In some embodiments, the photoreceptor is Phytochrome B (PhyB), and most preferably the first 650 amino-terminal amino acids of PhyB (i.e. HoloPhyB as set for in SEQ ID NO:2). In some embodiments, the photoreceptor protein can be PhyB derived from Arabidopsis thaliana. In some embodiments, the photoreceptor comprises an amino acid sequence that has at least 90% of identity with the amino acid sequence as set forth in SEQ ID NO:2

SEQ ID NO: 2
MVSGVGGSGGGRGGGRGGEEEPSSSHTPNNRRGGEQAQSSGTKSLRPRSNT
ESMSKAIQQYTVDARLHAVFEQSGESGKSFDYSQSLKTTTYGSSVPEQQIT
AYLSRIQRGGYIQPFGCMIAVDESSFRIIGYSENAREMLGIMPQSVPTLEK
PEILAMGTDVRSLFTSSSSILLERAFVAREITLLNPVWIHSKNTGKPFYAI
LHRIDVGVVIDLEPARTEDPALSIAGAVQSQKLAVRAISQLQALPGGDIKL
LCDTVVESVRDLTGYDRVMVYKFHEDEHGEVVAESKRDDLEPYIGLHYPAT
DIPQASRFLFKQNRVRMIVDCNATPVLVVQDDRLTQSMCLVGSTLRAPHGC
HSQYMANMGSIASLAMAVIINGNEDDGSNVASGRSSMRLWGLVVCHHTSSR
CIPFPLRYACEFLMQAFGLQLNMELQLALQMSEKRVLRTQTLLCDMLLRDS
PAGIVTQSPSIMDLVKCDGAAFLYHGKYYPLGVAPSEVQIKDVVEWLLANH
ADSTGLSTDSLGDAGYPGAAALGDAVCGMAVAYITKRDFLFWFRSHTAKEI
KWGGAKHHPEDKDDGQRMHPRSSFQAFLEVVKSRSQPWETAEMDAIHSLQL
ILRDSFKESEAAMNSKVVDGVVQPCRDMAGEQGIDELGAGTLEKLVDGAGS
WSHPQFEKENLYFQGLEHHHHHH

In some embodiments, the photoreceptor is immobilized in a solid surface. The term “solid surface” refers to a material having a rigid or semi-rigid surface. Such materials will preferably take the form of small beads, pellets, disks, chips, or wafers, although other forms may be used. The supports are generally made of conventional materials, e.g., plastic polymers, cellulose, glass, ceramic, stainless steel alloy, and the like. In some embodiments the solid support is a bead which is optionally labelled with one or more spectrally distinct fluorescent dyes, a number of methods of making and using sets of distinguishable beads have been described in the literature. These include beads distinguishable by size, wherein each size bead, beads with two or more fluorescent dyes at varying concentrations, wherein the beads are identified by the levels of fluorescence dyes, and beads distinguishably labelled with two different dyes, wherein the beads are identified by separately measuring the fluorescence intensity of each of the dyes. The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In some embodiments, the solid support is a magnetic bead that can be used use in magnetic separation. Typically, the magnetic bead is preferably made of a magnetic material selected from the group consisting of metals (e.g. ferrum, cobalt and nickel), an alloy thereof and an oxide thereof. In some embodiments, the partner is immobilized onto the support by any conventional method well known in the art. For instance, the partner that is directly or indirectly attached to the solid support is biotinylated and attached to the support via streptavidin, avidin or neutravidin. It thus contemplated that modified forms of avidin or streptavidin are employed to bind or capture the biotinylated partner. A number of modified forms of avidin or streptavidin that bind biotin specifically are known. Such modified forms of avidin or streptavidin include, e.g., physically modified forms (Kohanski, R. A. and Lane, M. D. (1990) Methods Enzymol. 194-200), chemically modified forms such as nitro-derivatives (Morag, E., et al., Anal. Biochem. 243 (1996) 257-263) and genetically modified forms of avidin or streptavidin (Sano, T., and Cantor, C. R., Proc. Natl. Acad. Sci. USA 92 (1995) 3180-3184).

In some embodiments, the optogenetic system of the present invention comprises a plurality of a recombinant proteins according to the invention specific for a receptor. In said embodiments, the photoreceptor protein may be unique or the system may comprise a plurality of photoreceptor protein, each being specific for a recombinant protein.

A further object of the present invention relates to a method of activating on demand a cell or a plurality of cells comprising i) contacting the cell or the plurality of cells with the optogenetic system of the present invention and ii) exposing the cell or the plurality of cells with a suitable wavelength of light wherein said exposition allows the oligomerization of the recombinant protein (e.g. Fab fragment) and thus triggering the activation of the cell or plurality of cells.

In some embodiments, the method further comprises a step of interrupting the activation on demand of the cell or the plurality of cells by exposing with a suitable wavelength of light wherein said exposition blocks the oligomerization of the recombinant protein (e.g. Fab fragment) and thus interrupts the activation of the cell or plurality of cells.

As used herein, the term “on-demand” refers to fact that the operator control finely and immediately the activation or deactivation of the cell or plurality of cells.

According to the present invention the cell can be of any type and may be selected from stem cell, progenitor cells, pluripotent stem cells, lineage-restricted stem cells, and somatic cells.

As used herein, a “stem cell” is a cell with the developmental potential to produce a more specialized cell type and at the same time to replicate itself. A stem cell may divide to produce two daughters that are themselves stem cells or it may divide to produce a daughter that is a stem cell and a daughter that is a more specialized cell type. A stem cell may originate from the embryo, fetus, or adult.

As used herein, a “progenitor cell” or “precursor cell” is a cell which occurs in fetal or adult tissues and is partially specialized. It divides and gives rise to differentiated cells.

As used herein, a “pluripotent stem cell” or “induced pluripotent stem cell” is a stem cell with the developmental potential to produce ectodermal cell types, mesodermal cell types, and endodermal cell types. An “embryonic stem cell” is a type of totipotent stem cell. That is, it is a cell that can give rise to every cell type in a mammal. A totipotent stem cell is a type of “pluripotent stem cell”.

As used herein, a “lineage-restricted stem cell” is a stem cell that can only give rise to cell types within one germ layer (i.e., to cell types within ectoderm or mesoderm or endoderm lineages). The lineage-restricted stem cell may have the potential to give rise to all cell types within the germ layer or it may only have the potential to give rise to a subset of cell types within the germ layer.

As used herein, a “somatic cell” is defined herein as a diploid cell of any tissue type that does not contribute to the propagation of the genome beyond the current generation of the organism. All cells except for germ cells are somatic cells and constitute the individual's body.

Suitable wavelengths of light for the activation or deactivation for the activation or deactivation of the photoreceptor protein, are known in the art for each particular photoreceptor protein. As an example, in case the photoreceptor protein is a phytochrome, activation can be effected by light having a wavelength of between 500 and 720 nm, preferably between 620 and 700 nm, and most preferably of about 650 nm. Further, deactivation can be effect by light having a wavelength of more than 720 nm, preferably of about 750 nm.

In some embodiments, the cell or plurality of cells is/are selected from neurons. As used herein, the term “neuron” refers to an animal cell consisting of a cell body, one of protrusions that extrude from the cell body, i.e., an axon or neurite, and several dendrites, and examples of the neuron may include sensory neurons, motoneurons, and interneurons. In addition, the neuron may include neurons constituting a central nervous system, a brain, brain stem, spinal cord and synaptic regions of the central nervous system and peripheral nervous systems, neurosustentacular cells, glia, and Schwann cells.

In some embodiments, the cell or plurality of cells is/are selected from immune cells.

As used herein, the term “immune cell” includes cells that are of hematopoietic origin and that play a role in the immune response. Immune cells include cells of the innate immune system and cells of the adaptive immune system. Immune cells include, for example, lymphocytes, such as B cells and T cells; natural killer cells; and myeloid cells, such as monocytes, macrophages, eosinophils, mast cells, basophils, and granulocytes.

In some embodiments, the immune cell is a cell of the innate immune system. The “innate immune system” is the nonspecific immune system that controls the body's response to an agent until the more specific adaptive immune system can produce specific antibodies and/or T cells (Modlin et al, N. Engl. J. Med 1999, 340: 1834-1835). The innate immune system generally involves phagocytic cells (e.g., neutrophils, monocytes, and macrophages); cells that release inflammatory mediators (e.g., basophils, mast cells, and eosinophils); natural killer cells (NK cells); and dendritic cells (DCs). In contrast, the “adaptive”, or “acquired, immune system”, is very specific in its responses. It is called an adaptive system because is occurs during the lifetime of an individual as an adaptation to infection with a pathogen. Adaptive immunity can be artificially acquired in response to a vaccine (antigens) or by administering antibodies, or can be naturally acquired by infection.

In some embodiments, the immune cell is an antigen presenting cell. As used herein, “antigen presenting cell” refers to cells that display foreign antigens complexed with major histocompatibility complexes (MHCs) on their surfaces, which are then recognized by T cells using their T cell receptors. Antigen presenting cells include cells that constitutively express MHC molecules (e.g., B lymphocytes, monocytes, dendritic cells, and Langerhans cells) as well as other antigen presenting cells that do not constitutively express MHC molecules (e.g., keratinocytes, endothelial cells, astrocytes, fibroblasts, and oligodendrocytes).

In some embodiments, the immune cell is a T cell. As used herein, the term “T cell” (i.e., T lymphocyte) is intended to include all cells within the T cell lineage, including thymocytes, immature T cells, mature T cells and the like, from a mammal (e.g., human). T cells include mature T cells that express either CD4 or CD8, but not both, and a T cell receptor. The various T cell populations described herein can be defined based on their cytokine profiles and their function. As used herein, the term “naive T cells” includes T cells that have not been exposed to cognate antigen and so are not activated or memory cells. Naive T cells are not cycling and human naive T cells are CD45RA+. If naive T cells recognize antigen and receive additional signals depending upon but not limited to the amount of antigen, route of administration and timing of administration, they may proliferate and differentiate into various subsets of T cells, e.g., effector T cells. As used herein, the term “effector T cell” includes T cells which function to eliminate antigen (e.g., by producing cytokines which modulate the activation of other cells or by cytotoxic activity). The term “effector T cell” includes T helper cells (e.g., Th1 and Th2 cells) and cytotoxic T cells. Th1 cells mediate delayed type hypersensitivity responses and macrophage activation while Th2 cells provide help to B cells and are critical in the allergic response (Mosmann and Coffman, 1989, Anna. Rev. Immunol. 7, 145-173; Paul and Seder, 1994, Cell 76, 241-251; Arthur and Mason, 1986, J. Exp. Med. 163, 774-786; Paliard et al., 1988, J. Immunol. 141, 849-855; Finkelman et al., 1988, J. Immunol. 141, 2335-2341). As used herein, the term “regulatory T cell” includes T cells which produce low levels of IL-2, IL-4, IL-5, and IL-12. Regulatory T cells produce TNFa, TGFp, IFN-γ, and IL-10, albeit at lower levels than effector T cells. Although TGFP is the predominant cytokine produced by regulatory T cells, the cytokine is produced at lower levels than in Th1 or Th2 cells, e.g., an order of magnitude less than in Th1 or Th2 cells. Regulatory T cells can be found in the CD4+CD25+ population of cells (see, e.g., Waldmann and Cobbold. 2001. Immunity. 14:399). Regulatory T cells actively suppress the proliferation and cytokine production of Th1, Th2, or naive T cells which have been stimulated in culture with an activating signal (e.g., antigen and antigen presenting cells or with a signal that mimics antigen in the context of MHC, e.g., anti-CD3 antibody plus anti-CD28 antibody). As used herein, the term “exhausted T cell” refers to malfunctional T cells that are characterized by the stepwise and progressive loss of T-cell functions and can culminate in the physical deletion of the responding cells. Exhausted T cell may arise during chronic infections and cancer. For example, exhaustion is well-defined during chronic lymphocytic choriomeningitis virus infection and commonly develops under conditions of antigen-persistence, which occur following many chronic infections that are of significant public health concern including hepatitis B virus, hepatitis C virus and human immunodeficiency virus infections, as well as during tumor outgrowth (see. e.g., John Wherry, Nature Immunology 12, 492-499, 2011). As used herein, the term “anergic T cell” refers to T cells that are functionally inactivated and unable to initiate a productive response even when antigen is encountered in the presence of full co-stimulation (see, e.g., Macian F. et al, Curr Opin Immunol. 2004, 16(2):209-16.) T cell anergy is a tolerance mechanism in which the lymphocyte is intrinsically functionally inactivated following an antigen encounter, but remains alive for an extended period of time in a hyporesponsive state. Models of T cell anergy affecting both CD4+ and CD8+ cells fall into two broad categories. One, clonal anergy, is principally a growth arrest state, whereas the other, adaptive tolerance or in vivo anergy, represents a more generalized inhibition of proliferation and effector functions (see, e.g., Schwartz RH. Annu Rev Immunol. 2003; 21:305-34).

In some embodiments, the plurality of cells is encompasses in a tissue, an organ or an organism. Accordingly, the method of the present invention can be applied to in vitro or in vivo system.

As used herein, the term “tissue” as used herein refers to any type of tissue in human or animals, and includes, but is not limited to, vascular tissue, skin tissue, hepatic tissue, pancreatic tissue, neural tissue, urogenital tissue, gastrointestinal tissue, skeletal tissue including bone and cartilage, adipose tissue, connective tissue including tendons and ligaments, amniotic tissue, chorionic tissue, dura, pericardia, muscle tissue, glandular tissue, facial tissue, ophthalmic tissue.

In some embodiments, the tissue is a tumor tissue. As used herein, the term “tumor tissue” means both tissue known to contain a tumor and tissue believed to contain a tumor. Here, the term “tumor” comprises both benign tumors and malignant tumors. In particular, the term “tumor” comprises cancers and, in particular, metastasizing cancers and carcinomas.

As used herein the term “organ” refers to a solid vascularized organ that performs a specific function or group of functions within an organism. The term organ includes, but is not limited to heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus.

As used herein, the term “organism” refers to any living creature capable of reproduction. In some embodiments, the organism is a mammal. The term “mammal” refers preferably, but is not limited to, to such organisms as rodents, ungulates, primates, mice, rats, rabbits, guinea pigs, horses, sheep, pigs, goats, and cows, more preferably to cats, dogs, monkeys, and apes, and most preferably to humans.

In some embodiments, the intensity of light to which the cell or the plurality of cells is exposed can be used to control the extent of the activation. For example, low-intensity red light will achieve only partial, titrated association. Total illumination doses less than 1,000 micromoles of photons per square meter can be regarded as low intensity red light. Total illumination doses greater than 10,000 micromoles of photons per square meter can be regarded as high-intensity light that is sufficient for 100% conversion. The intensity of red light required to convert a significant fraction or majority or substantially all the photoreceptor to an activated state can be empirically.

In some embodiments, the time of exposure to light can be varied according to effect needed and light intensity chosen, e.g., for about 1, 10 or 100 milliseconds, or about 1, 5 or 10 seconds, or about 1, 2, 3, 5, 10, 20 or 30 minutes, or about 1, 2, 3 or 5 hours, or about 1, 2, 3, or 5 days, or 1, 2 or 3 weeks. In some embodiments, the cell or plurality of cells is/are exposed for a short time. For example, the cell or plurality of cells can be exposed to ref or infra-red light for less than a minute, e.g., about 1, 5, 10, 20 or 40 seconds. The light can be delivered by known devices such as a laser, in one or more pulses or individual portions. For example, a UV-pumped red dye cell laser can shoot ultrafast pulses of light that last about 5 ns; these can be applied, e.g., at low intensity at about 20 Hz for about 5 s to minutes.

The method of the present invention allows modulating temporarily the activation of the cell or plurality of cells. The method disclosed herein can indeed allow extremely quick activation of the cell or plurality of cells. Accordingly, the method of the present invention allows control of activation of the cell or the plurality of cells within 1 minute, or sometimes within 10-20 seconds, and sometimes even within one second.

The method of the present invention also allow modulation spatially the activation of the cell or plurality of cells. Said activation can be locally triggered especially and thus can be restricted to a particular tissue, organ or organism. For example a portion of a tissue, organ or organism can be exposed to “activating” red light that induces the agonistic activity of the recombinant protein (e.g. Fab fragment). In another example, the tissue, organ or organism can be bathed in continuous “inactivating” infrared light, while a localized beam of activating red light is restrictively delivered to a specific portion the tissue, organ or organism, resulting in well-defined localization.

The method of the invention may thus find various application. One particular application of interest is to modulating an immune response in a tissue, organ or organism. As used herein, the term “modulating an immune response” as used herein means that the substance evokes a change in an immune response and includes an increase or enhancement of the immune response as well as a decrease or suppression in the immune response. In particular, the method of the present invention is particularly suitable to modulating a T cell response. As used herein, the term “modulating a T cell response” as used herein means that the substance evokes a chance in a T cell response and includes an increase or enhancement of the T cell response as well as a decrease or suppression in the T cell response.

Thus the method of the present invention may particularly suitable for the treatment of cancer or inflammatory auto-immune diseases.

As used herein, the term “cancer” has its general meaning in the art and includes, but is not limited to, solid tumors and blood borne tumors The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyo sarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the autoimmune inflammatory disease is selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospho lipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

A further object relates to a kit or device comprising the recombinant proteins, protein constructs, nucleic acids, cells, reagents or materials of the invention or any combination thereof. In some embodiments, the kit or device comprises the optogenetic system of the present invention. Typically, the kit or device of the present invention may further comprise at least one light source. Typically, the light source is a laser. For instance, one light source for local activation can be a nitrogen pulsed UV dye-cell laser exciting a Rhodamine 650 dye which emits at 650 nm. One could also use any other laser with laser lines in the red region of the spectrum, such as 750 nm to allow inactivation of the system. In some embodiments, the light source for spatially controlled activation and deactivation can be any system that uses a computer controlled spatial light modulator to project the light source onto the cell or plurality of cells. In some embodiments, the device is a microscope so that the laser beam may be guided optically to be aligned with the imaging axis of the microscope and to center the laser spot directly onto the sample along the Z dimension of this axis. The kit optionally contains instructions that instruct a user to introduce proteins, protein constructs, nucleic acids, and/or reagents of the invention and/or to regulate the activation of the cell or plurality of cells by modulating exposure to light (e.g., red and/or infrared light).

The invention will be further illustrated by the following figures and examples.

However, these examples and figures should not be interpreted in any way as limiting the scope of the present invention.

### FIGURES

FIG. 1: Representation of the H57OptoFab system involving PHYB/PIF Optogenetic module. A. PIF6 peptide is cloned at C terminus of the H57 Fab Heavy chain. 650 nm wavelength light triggers the binding of the OptoFab to PHYB. This process is reversed following a 730 nm wavelength light. B. The H57 OptoFab system allows the light dependent immobilization of the TCR allowing an accurate control of TCR signaling.

FIG. 2: Western blot analysis of OptoFab production. Affinity purified proteins from the supernatant of OptoFab transfected HEK cells analyzed by western blot under reducing condition A. Or non-reducing conditions B. (FT: column flow-through, Elution: eluted proteins).

FIG. 3: Analysis of the H57 OptoFab specificity for TCR. A. Murine primary CD4 T cells were incubated or not with H57 OptoFab, then labelled with anti-His Alexa647 and analyzed by cytometry. B. Murine primary CD4 T cells were incubated with the indicated dose of the H57 OptoFab, then labelled with anti-His Alexa647 and analyzed by cytometry. The graph shows the quantity of protein versus the MFI. C. Competition experiment between a recombinant H57 Fab Alexa 488 and the H57 OptoFab.

FIG. 4: Analysis of HoloPhyB purity and functionality. A. SDS-PAGE analysis of affinity purified HoloPhyB: MW: Molecular Weight, FT: Flow Through, Elution: Eluted HoloPhyB. B. Absorption spectrum of purified HoloPhyB kept in dark (dotted black curve), illuminated with a 656 nm wavelength light (curve 1) and illuminated with a 656 nm wavelength light followed by a 740 nm wavelength light (curve 2).

FIG. 5: H57 OptoFab bind to HoloPhyB in a light dependent manner. Pull-Down experiment of H57-OptoFab binding to HoloPhyB-coated beads. Upper panel: western blot analysis of the pulled-down proteins following different illumination patterns. Lower panel: signal quantification using Fiji software.

FIG. 6: H57 OptoFab allows the light-controlled stimulation of murine primary T lymphocytes. Primary T lymphocytes loaded with PBDX Calcium indicator and incubated with H57 OptoFab were dropped on a HoloPhyB coated cover slip and exposed to the indicated light wavelengths. A. Microscope field containing primary T cells exposed to a 740 nm wavelength light (left panel), then to a 656 nm wavelength light (right panel). B. Quantification of calcium indicator fluorescence in live primary T cells during specific light exposure. Cell fluorescence has been quantified over time using FIJI software. Each symbol correspond to an individual cell.

### EXAMPLE 1: METHODS

Generation of the H57-PIF6 OptoFab:

pYD7-HC-PIF (HC=fragment of the Heavy Chain derived from the monoclonal antibody H57) plasmid and pTT22-LC (LC=Light Chain derived from the monoclonal antibody H57) were cloned using In-Fusion HD cloning kit (Clonetech) in a standard reaction mixture. Oligonucleotide were synthesized by Sigma Aldrich. PCR templates are listed in table 3. PCR-amplified fragments were purified following the manufacturer's instructions. Host vector backbone was linearized using restriction enzyme. All cloning products were confirmed by sequencing (Eurofins Genomics).

H57 F(ab)-Pif was produced by cotransfecting HEK293T ebna cells with 10 μg of pYD7-HC-PIF plasmid and 30 μg of pTT22-LC plasmid (ratio 1:3) using Polyethylenimine. Cells were then maintained in DMEM high glucose (GIBCO) 2% FBS+0.5% Tryptone TN1+1.25 mM valproic acid supplemented with G418 at 37° C. with 5% CO2 in a humidified incubator. Supernatants were collected 7 days later and the recombinant OptoFab was purified by Ni-NTA affinity chromatography. Production of F(ab)-PIF was verified by western blotting.

Production of HoloPhyB:

HoloPhyB-StrepTag-pet28a was obtained by site-directed mutagenesis. StrepTag was added inside HoloPhyB-pet28a plasmid using QuickChange II XL Site-Directed Mutagenesis Kit (Agilent Technologies) following the manufacturer's instructions. Oligonucleotides were synthesized by Sigma Aldrich.

To produce HoloPhyB protein, E. coli cells BL21 were transformed with a combination of one plasmid encoding for PΦB synthase lacking transit peptide (AHY2), and a second plasmid driving heme oxygenase-1 expression and a third plasmid for HoloPhyB synthesis (HoloPhyB-strepTag expression) (Leung et al., 2008).

Single colonies were selected and grown in synthetic complete medium (LB broth with kanamycin) overnight at 37° C. Production was induced using IPTG at OD600 of 0.4-0.6 and kept overnight at 17° C. Proteins was extracted using a lysis buffer (Tris 50 mM+NaCl 300 mM+Imidazole 10 mM+lysozyme 0.25 mg/ml-pH 8) with DNase and MgSO4. Finally, proteins were purified by Ni-NTA affinity chromatography. Production of HoloPhyB was verified by western blotting.

TABLE 1
PCR templates

| Inserts | Forward (5′-3′) | Reverse (5′-3′) |
|---|---|---|
| LC | gatctctagcgaattcatgaaatacctattgcctacggc | ggccgctagcaagctttcagcactcgccc |
| | agccgctggattgttattactcgcggcccagccggccat | ctgttgaa |
| | ggcctatgagctgatacagccttcc | |
| HC | gcgaattccctctagaatggagtttgggctgagctgggt | cggcctcgagcggccgcttaatgatggtg |
| | tttcctcgttgctctttttagaggtgtccagtgtgaggt | atgatgatagaaccggagccggtcttgtc |
| | gtacttggtggaatctgg | acagctcttggg |
| PIF | tgacaagaccggcgccggtagcggcagtggtagtggta | ggcctcgagcggcgccggggatccttaat |
| | | gatggtgat |
| StrepTag | cgacggcgccggatcctggagccacccgcaatttgaaa | tgaaaatacaggttttctttttcaaattg |
| | aagaaaacctgtattttca | cgggtggctccaggatccggcgccgtcg |

Photoconversion and Absorption Spectrum Analysis:

The photoconversions of HoloPhyB were assessed using BioLED light source at 656 nm or 740 nm for the indicated times. The absorption spectrum of holoPhyB was determined between 260 nm and 700 nm using a spectrophotometer (NanoDrop).

Measurement of the OptoFab Binding to TCR Using Flow Cytometry

Murine CD4+ T lymphocytes were incubated with H57 OptoFab diluted in PBS containing 2% SVF 1h at 10° C. Then after 2 washes, cells were incubated with an Alexa647 conjugated anti-his antibody (BD-Pharmingen) 30 minutes at 4° C. The binding of the H57 OptoFab was measured using a flow cytometer (BD FACSCanto-BD Biosciences)

Pull-Down Assay

HoloPhyB has been attached to strep-tactin coated beads (ferrimagnetic agarose beads coupled to the Strep-Tactin® IBA) in PBS containing 0.1% Triton X-100, 5 mM β-mercaptoethanol, and 1 mm PMSF). Then, soluble OptoFab has been added to the beads and exposed to the different pattern of light. Samples were then washed three times and D-Biotin was added to elute PhyB. The amount of OptoFab interacting with PhyB in each condition was assessed by western blot. The measurement and quantification of the western blot has been performed using a CCD camera (Azure system).

T Cell Stimulations and Ca2+ Influx Analyses:

Murine primary CD4 T cells has been loaded with PBDX Calcium dye (Sigma) 1 h at 37° C., then incubated with H57 OptoFab 1 h at 10° C. After 3 washes, T cells are then dropped into HoloPhyB-coated glass bottom LabTek chambers at 37° C. and imaged using a videomicroscope (Zeiss). BioLED illumination system has been plugged in the white-light path and exposure to 646 nm or 740 nm lights are set using BioLED light source control Module from Mightex. Quantifications has been performed using Fiji software.

### EXAMPLE 2: THE OPTOFAB SYSTEM

OptoFab is a recombinant molecular system allowing the accurate control of the agonistic properties of an Antibody-derived Fab fragment in time and in space using specific wavelengths of light. It consists in a Fab fragment derived from an antibody of interest, linked to optogenetic modules that confer a light response capacity. Indeed, antibody derived Fab fragments generally keep the specificity of the antibody for its epitope, but not its agonistic properties. However, when Fab fragments are immobilized or oligomerized, they recover the agonistic properties of the whole antibody. These characteristics that are shared by numerous Fab fragments derived from agonistic antibodies, are at the basis of the OptoFab concept as its objective is to manipulate the oligomerization/immobilization statue of a Fab fragment using optogenetics to control its agonistic property. Optogenetics is a rising technology that consist in using light sensitive domains from plant or prokaryote proteins to control with light biological processes such as protein-protein interactions (Repina et al., 2017). Optogenetics provide the control of biological process with a temporal resolution in the millisecond range and a spatial resolution above the micrometer scale (restricted by the light diffraction limit).

We first generated an OptoFab system allowing to reversibly control T lymphocyte activation using specific wavelengths of light. Thus, we generated a recombinant protein composed of i) a Fab fragment derived from the H57 monoclonal antibody that recognizes an epitope in the β-chain of the TCR and has agonistic properties that drive T cell activation, coupled to ii) the Phytochrome Interacting Factor 6 (PIF6). The Phytochrome B of Arabidopsis Thaliana, when exposed to a light at 650 nm, experience a conformational change leading to the opening of a binding site for PIF6. An exposure to light at 730 nm reverses this process and free PIF6 (Leung et al., 2008; Toettcher et al., 2013).

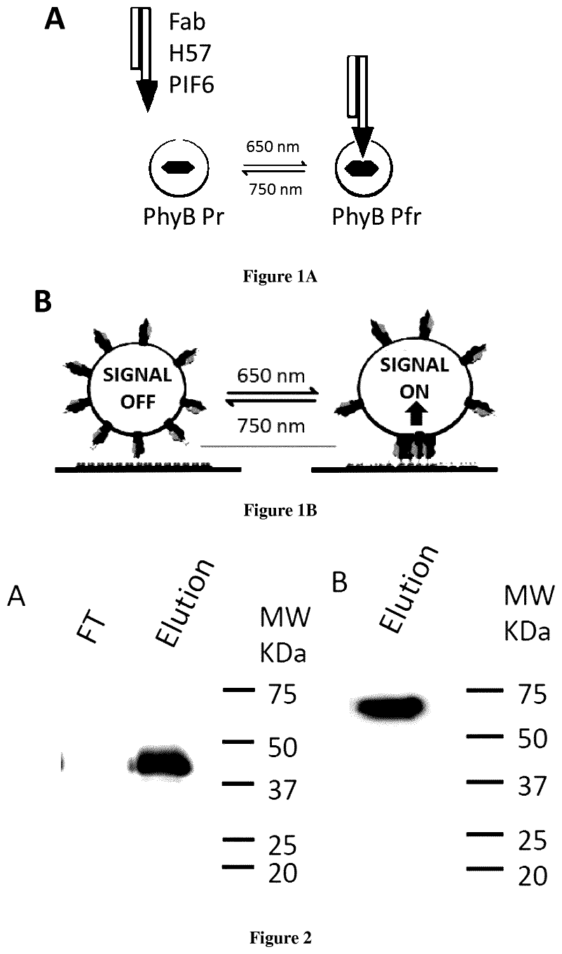

The idea here is to use light to accurately control in time and space the agonistic property of the H57 Fab fragment by inducing is aggregation/immobilization on surfaces coated with recombinant Phy-B derived molecule (FIG. 1A). By this way, we aim to drive the capture of TCR and thus, T cell stimulation (FIG. 1B).

Phytochrome Interacting Factor 6 Peptide does not Alter Fab Fragment Derived from H57 Specificity:

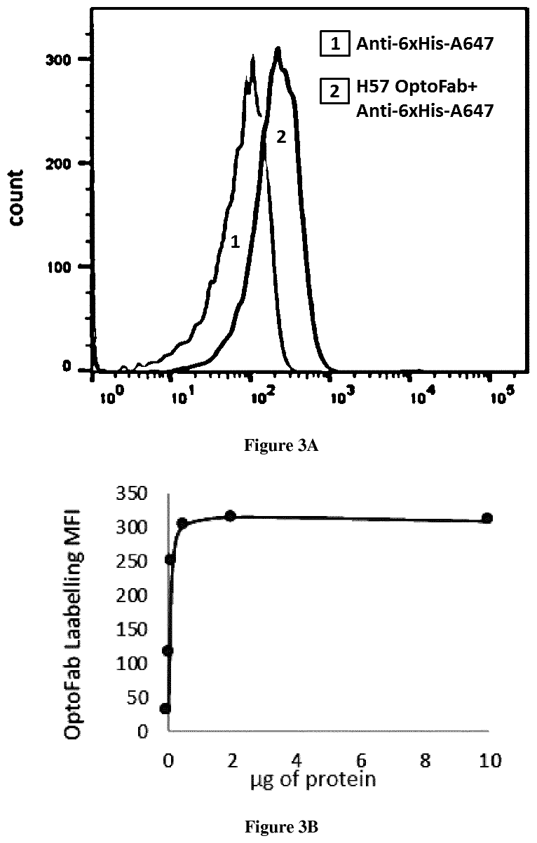

We wanted to generate a Fab fragment derived from H57 monoclonal antibody that can be immobilized or clustered using light. We thus decided to clone a sequence encoding for the Phytochrome Interacting Factor 6 peptide (PIF6) at the 3′ end of the fragment of the H57 heavy chain forming the Fab fragment. The goal is to generate a Fab fragment whose immobilization or clustering can be controlled with light through its interaction with Phytochrom B (FIG. 1). We co-transfected this construct with a plasmid encoding for H57 Fab light chain in HEK cells to produce a PIF6 modified H57 Fab (optoFab) (FIG. 1). The optoFab has been affinity purified on Ni column thanks to a 6×His tag added at the carboxy-terminal end of PIF6. Western blot analysis of the purified OptoFab in reducing condition showed that, as expected, the modified heavy chain migrate at 41 kDa (FIG. 2A). Under non-reducing condition, the OptoFab migrates at 67 kDa (FIG. 2B). This shift in the migration is due to the interaction of the heavy chain with the light chain that is preserved under non-reducing condition. This data suggested that the purified OptoFab is correctly folded and is composed of one fragment of H57 heavy chain-PIF6, and one light chain fragment. PIF6 peptide doesn't seem to alter H57 Fab folding and secretion.

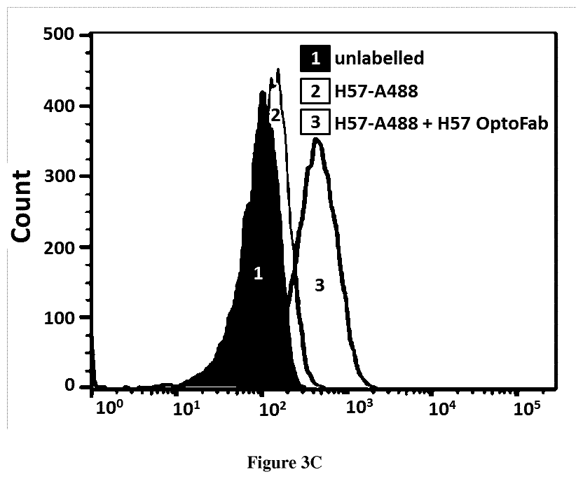

To go further in optoFab functionality analyses, we tested if the OptoFab derived from H57 antibody kept its specificity for the TCRβ chain. To test if the H57 OptoFab maintain its specificity for TCR, T cells have been incubated with a solution containing 2 ug/mL of OptoFab, 1 h at 10° C., then labelled with an anti-6×His antibody coupled to Alexa647 and analyzed by flow cytometry (FIG. 3). We observed a labelling of T cell similar to the one obtained with a fluorescent H57 Fab fragment. We then performed competition experiments to verify that the H57 OptoFab has the same specificity than the original H57 Fab. A 10-fold excess of OptoFab have been added on cells incubated with H57 Fab coupled to Alexa 488. This treatment fully abrogates the fluorescent H57 Fab labelling, showing that OptoFab competed with the native Fab for TCR binding. These experiments show that the H57-derived OptoFab is specific for TCR β chain.

Production of Functional Recombinant HoloPhyB:

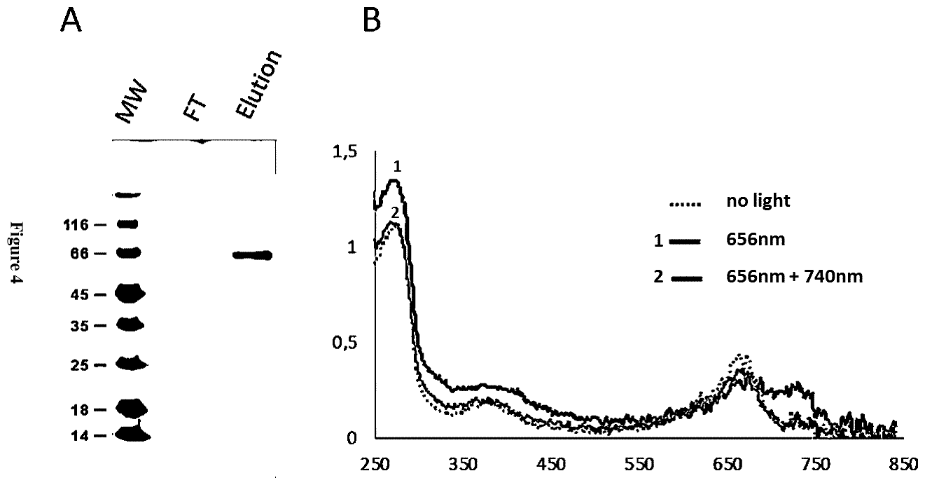

To capture the OptoFab, we decided to produce a truncated form of PhyB containing the 1-650 aminoacids named Holo-PHYB (Leung et al., 2008). We cloned a single StrepTag followed by a 6×His Tag at the carboxy-terminal end of Holo-PHYB. This protein has been co-transfected in BL21 bacteria with a plasmid encoding Heme Oxygenase 1 and a plasmid encoding PΦB synthase lacking the transit peptide (Leung et al., 2008). These two constructs generate a PΦB adduct in the bacteria that favour the production of soluble HoloPhyB complexed with PΦB chromophore, which is required for its functionality. HoloPhyB was then affinity purified on Ni column, followed by a second step of purification using ion exchange column. As expected, a SDS-PAGE followed by a Coomassie staining analysis showed that the purified protein migrated at 75 kDa (FIG. 4A). A characteristic of the complex HoloPhyB-PΦB is its absorption spectrum. A spectral analysis showed that the protein we purified had two absorption peaks, one at 280 nm, and another at 660 nm (FIG. 4B), indicating that we succeed in producing the HoloPhyB-PΦB complex. When functional, this complex can display two distinct spectrums. In its Pr form, it absorbs red-light (at 665 nm) as shown in FIG. 4B. But following red light exposure, the complex switch in its Pfr form and can absorb Far-red light. As shown in FIG. 4B, a 5 minutes exposure of the purified HoloPhyB-PΦB complex to a 656 nm red light modified its absorption spectrum with the appearance of a peak around 730 nm (far-red light). Interestingly, a 740 nm far-red light reverse this phenomenon inducing a return to the Pr form. Altogether, these data show that we produced a photo-convertible functional HoloPhyB-PΦB complex.

The H57 Derived OptoFab Interacts Specifically with HoloPhyB-PΦB Complex in a Light Dependent Manner:

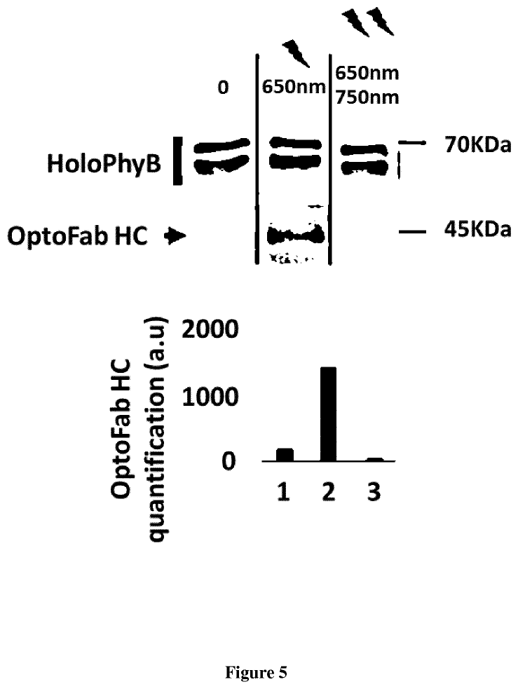

Soluble H57 Fab binds to the TCR without triggering any signalling. However, when immobilized on a planar surface or on beads, it triggers TCR signalling (data not shown). To verify if H57 OptoFab can be used as a molecular switch to control T cell activation, we next tested if the OptoFab can be specifically and reversibly immobilized with appropriate light wavelength. HoloPhyB has been coated on streptactin-coated beads, then incubated with OptoFab and exposed to different pattern of lights before a pull-down experiment (FIG. 5). As shown by western blot, when the system has been kept in the dark, the OptoFab is not pulled-down with HoloPhyB-coated beads. However, when the system is exposed to a 656 nm wave length light during 5 minutes, we observed that the OptoFab is pulled-down with HoloPhyB-coated beads. This data shows that an exposure to a red light triggers the binding of the OptoFab to HoloPhyB. Furthermore, when the system was exposed to a 740 nm wavelength light or kept 1 hour in the dark following the exposure to the red light, the interaction between the OptoFab and HoloPhyB is disrupted. All together, these data show that the light responding module of the recombinant proteins we produced is functional. The H57-derived OptoFab can be reversibly immobilized “on demand” using different wavelength of light.

H57 Derived OptoFab Acts as a Cellular On-Off Switch for T Cells:

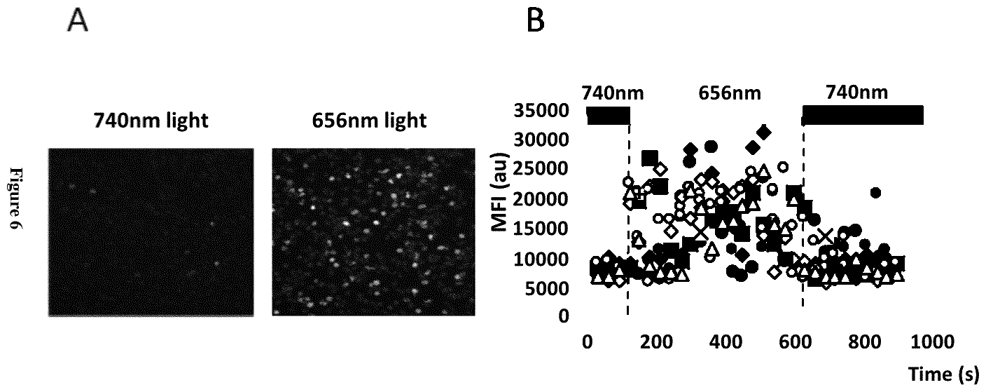

Considering that the H57-derived OptoFab is specific for TCR and can be immobilized with light, we then wanted to test if it could allow a time gated control of TCR stimulations. We first verified that as the unmodified H57 Fab, the H57-derived OptoFab does not triggers TCR signalling per se. A very sensitive way to detect TCR signalling under a microscope is the analysis of Ca^{t+} influx in live cells using specific Ca^{t+} probes. Naïve primary T cells loaded with the calcium indicator PBDX have been incubated in the presence of 1 ug/mL of OptoFab, were dropped on a glass surface coated with holoPhyB and analysed under the microscope (FIG. 6A). When the system was exposed to a 740 nm wavelength light, no Ca^{t+} influxes were detected in the T cells, indicating that they remained unstimulated. However, a 656 nm wavelength light exposure triggered calcium influxes a few seconds later, indicating that the light dependent immobilization of the OptoFab driven by HoloPhyB triggered TCR stimulation. A consecutive exposure to a 730 nm wavelength light induced a rapid decrease of Ca^{t+} concentration in T cells, showing that TCR stimulation has been interrupted (FIG. 6B). Altogether, these data showed that the H57 OptoFab module allows a time gated control of TCR triggering. It acts as a molecular on/off switch for T lymphocytes.

### EXAMPLE 3: OPTOFAB AND HOLOPHYB SEQUENCES

H57 OptoFab

H57 Heavy Chain-PIF:
SEQ ID NO: 3
MEFGLSWVFLVALFRGVQCEVYLVESGGDLVQPGSSLKVSCAASGFTFSDF
WMYWVRQAPGKGLEWVGRIKNIPNNYATEYADSVRGRFTISRDDSRNSIYL
QMNRLRVDDTAIYYCTRAGRFDHFDYWGQGTMVTVSSASTKGPSVFPLAPS
SKSTSGGTAALGCLVKDYFPEPVTVSWNSGALTSGVHTFPAVLQSSGLYSL
SSVVTVPSSSLGTQTYICNVNHKPSNTKVDKRVEPKSCDKTGAGSGSGSGS
GSMMFLPTDYCCRLSDQEYMELVFENGQILAKGQRSNVSLHNQRTKSIMDL
YEAEYNEDFMKSIIHGGGGAITNLGDTQVVPQSHVAAAHETNMLESNKHVD
GSGSGSGSGSENLYFQGHHHHHH*
H57 Light Chain :
SEQ ID NO: 4
MKYLLPTAAAGLLLLAAQPAMAYELIQPSSASVTVGETVKITCSGDQLPKN
FAYWFQQKSDKNILLLIYMDNKRPSGIPERFSGSTSGTTATLTISGAQPED
EAAYYCLSSYGDNNDLVFGSGTQLTVLRGRTVAAPSVFIFPPSDEQLKSGT
ASVVCLLNNFYPREAKVQWKVDNALQSGNSQESVTEQDSKDSTYSLSSTLT
LSKADYEKHKVYACEVTHQGLSSPVTKSFNRGEC*
HoloPhyB:
SEQ ID NO: 2
MVSGVGGSGGGRGGGRGGEEEPSSSHTPNNRRGGEQAQSSGTKSLRPRSNT
ESMSKAIQQYTVDARLHAVFEQSGESGKSFDYSQSLKTTTYGSSVPEQQIT
AYLSRIQRGGYIQPFGCMIAVDESSFRIIGYSENAREMLGIMPQSVPTLEK
PEILAMGTDVRSLFTSSSSILLERAFVAREITLLNPVWIHSKNTGKPFYAI
LHRIDVGVVIDLEPARTEDPALSIAGAVQSQKLAVRAISQLQALPGGDIKL
LCDTVVESVRDLTGYDRVMVYKFHEDEHGEVVAESKRDDLEPYIGLHYPAT
DIPQASRFLFKQNRVRMIVDCNATPVLVVQDDRLTQSMCLVGSTLRAPHGC
HSQYMANMGSIASLAMAVIINGNEDDGSNVASGRSSMRLWGLVVCHHTSSR
CIPFPLRYACEFLMQAFGLQLNMELQLALQMSEKRVLRTQTLLCDMLLRDS
PAGIVTQSPSIMDLVKCDGAAFLYHGKYYPLGVAPSEVQIKDVVEWLLANH
ADSTGLSTDSLGDAGYPGAAALGDAVCGMAVAYITKRDFLFWFRSHTAKEI
KWGGAKHHPEDKDDGQRMHPRSSFQAFLEVVKSRSQPWETAEMDAIHSLQL
ILRDSFKESEAAMNSKVVDGVVQPCRDMAGEQGIDELGAGTLEKLVDGAGS
WSHPQFEKENLYFQGLEHHHHHH*

### REFERENCES

Throughout this application, various references describe the state of the art to which this invention pertains. The disclosures of these references are hereby incorporated by reference into the present disclosure.
Leung, D. W., Otomo, C., Chory, J., and Rosen, M. K. (2008). Genetically encoded photoswitching of actin assembly through the Cdc42-WASP-Arp2/3 complex pathway. Proc Natl Acad Sci USA 105, 12797-12802.
Repina, N. A., Rosenbloom, A., Mukherjee, A., Schaffer, D. V., and Kane, R. S. (2017). At Light Speed: Advances in Optogenetic Systems for Regulating Cell Signaling and Behavior. Annu Rev Chem Biomol Eng 8, 13-39.
Toettcher, J. E., Weiner, O. D., and Lim, W. A. (2013). Using optogenetics to interrogate the dynamic control of signal transmission by the Ras/Erk module. Cell 155, 1422-1434.

1. A recombinant protein comprising a variable domain of an antibody that is fused at its c-terminal end to a factor that can interact with a photoreceptor protein in a light-dependent manner.

2. The recombinant protein of claim 1 wherein the variable domain is selected from the group consisting of VH domains, VL domains, Of and single domain antibodies (sdAbs).

3. The recombinant of claim 1 which comprises a Fab fragment wherein the VH domain of the Fab fragment is fused at its c-terminal end to the factor that can interact with a photoreceptor protein in a light-dependent manner.

4. The recombinant protein of claim 3 wherein the Fab fragment derives from an agonistic antibody.

5. The recombinant protein of claim 4 wherein the agonistic antibody is specific for a receptor of an immune cell.

6. The recombinant protein of claim 5 wherein the agonistic antibody is specific for a costimulatory receptor selected from the group consisting of CD134 (OX40), CD137 (4-1BB), CD28, GITR, CD27, CD70, ICOS, RANKL, TNFRSF25 (DR3), CD258 (LIGHT), CD40 and HVEM.

7. The recombinant protein of claim 1 wherein the factor is selected from the group consisting of Phytochrome Interacting Factors (PIFs), FHY1/FHL, Phytochrome kinase substrate 1 (PKS1), nucleoside diphosphate kinase 2 (NDPK2), cryptochromes, Aux/IAA proteins, phosphatases, E3 ubiquitin ligases, and ARR4.

8. The recombinant protein of claim 1 wherein the factor is selected from the group consisting of PIF1, PIF2, PIF3, PIF4, PIF5, PIF6, and PIF7.

9. The recombinant protein of claim 1 wherein the factor comprises an amino acid sequence that has at least 90% identity with the amino acid sequence as set forth in SEQ ID NO:1.

10. A nucleic acid encoding for the recombinant protein of claim 1.

11. A host cell transformed with the nucleic acid of claim 10.

12. An optogenetic system comprising at least one recombinant protein of claim 1 and at least one photoreceptor protein.

13. The optogenetic system of claim 2 wherein the at least one photoreceptor protein is a phytochrome selected from the group consisting of Phytochrome A (PhyA), Phytochrome B (PhyB), Phytochrome C (PhyC), Phytochrome D (PhyD), and Phytochrome E (PhyE).

14. The optogenetic system of claim 13 wherein the at least one photoreceptor protein comprises an amino acid sequence that has at least 90% identity with the amino acid sequence as set forth in SEQ ID NO:2.

15. The optogenetic system of claim 12 wherein the at least one photoreceptor protein is immobilized on a solid surface.

16. A method of activating on demand a cell or a plurality of cells comprising i) contacting the cell or the plurality of cells with the optogenetic system of claim 12 and ii) exposing the cell or the plurality of cells to a wavelength of light sufficient to oligomerize the at least one recombinant protein and activate the cell or the plurality of cells.

17. The method of claim 16 wherein the cell or the plurality of cells is embedded in a tissue, organ or organism.

18. The method of claim 16 wherein the cell or the plurality of cells are lymphocytes; natural killer cells; or myeloid cells.

19. A method of modulating an immune response in a tissue, organ or organism comprising,
i) contacting a cell or a plurality of cells in the tissue, organ or organism with the optogenetic system of claim 12, and
ii) exposing the cell or the plurality of cells to a wavelength of light sufficient to oligomerize the at least one recombinant protein and modulate the immune response.

20. A method of treating cancer or an inflammatory auto-immune disease in a subject in need thereof, comprising
i) contacting a cell or a plurality of cells in a tissue, organ or organism of the subject with a therapeutically effective amount of the optogenetic system of claim 12, and
ii) exposing the cell or the plurality of cells to a wavelength of light sufficient to oligomerize the at least one recombinant protein, modulate the immune response of the subject and treat the cancer or the inflammatory auto-immune disease.

21. The recombinant protein of claim 7 wherein
the cryptochrome is CRY1 or CRY2,
the phosphatase is FyPP or PAPP5, and
the E3 ubiquitin ligase is COP1.

22. The method of claim 16 wherein the recombinant protein comprises a Fab fragment.

23. The method of claim 18, wherein
the lymphocytes are B cells and/or T cells; and
the myeloid cells are monocytes, macrophages, eosinophils, mast cells, basophils, or granulocytes.


SEQUENCE LISTING (12 sequences)

SEQ ID NO: 1 - protein, 116 aa, Arabidopsis thaliana
MFLPTDYCCRLSDQEYMELVFENGQILAKGQRSNVSLHNQRTKSIMDLYEAEYNEDFMKSIIHGGGGAITNLGDTQVVPQSHVAAAHETNMLESNKHVDGSGSGSGSGSENLYFQG

SEQ ID NO: 2 - protein, 686 aa, Arabidopsis thaliana
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAGTLEKLVDGAGSWSHPQFEKENLYFQGLEHHHHHH

SEQ ID NO: 3 - protein, 380 aa, Artificial
  Synthetic H57 heavy chain
MEFGLSWVFLVALFRGVQCEVYLVESGGDLVQPGSSLKVSCAASGFTFSDFWMYWVRQAPGKGLEWVGRIKNIPNNYATEYADSVRGRFTISRDDSRNSIYLQMNRLRVDDTAIYYCTRAGRFDHFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGAGSGSGSGSGSMMFLPTDYCCRLSDQEYMELVFENGQILAKGQRSNVSLHNQRTKSIMDLYEAEYNEDFMKSIIHGGGGAITNLGDTQVVPQSHVAAAHETNMLESNKHVDGSGSGSGSGSENLYFQGHHHHHH

SEQ ID NO: 4 - protein, 238 aa, Artificial
  Synthetic H57 light chain
MKYLLPTAAAGLLLLAAQPAMAYELIQPSSASVTVGETVKITCSGDQLPKNFAYWFQQKSDKNILLLIYMDNKRPSGIPERFSGSTSGTTATLTISGAQPEDEAAYYCLSSYGDNNDLVFGSGTQLTVLRGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 103 bp, Artificial
  Synthetic primer
gatctctagcgaattcatgaaatacctattgcctacggcagccgctggattgttattactcgcggcccagccggccatggcctatgagctgatacagccttcc

SEQ ID NO: 6 - DNA, 37 bp, Artificial
  Synthetic primer
ggccgctagcaagctttcagcactcgcccctgttgaa

SEQ ID NO: 7 - DNA, 96 bp, Artificial
  Synthetic primer
gcgaattccctctagaatggagtttgggctgagctgggttttcctcgttgctctttttagaggtgtccagtgtgaggtgtacttggtggaatctgg

SEQ ID NO: 8 - DNA, 70 bp, Artificial
  Synthetic primer
cggcctcgagcggccgcttaatgatggtgatgatgatagaaccggagccggtcttgtcacagctcttggg

SEQ ID NO: 9 - DNA, 38 bp, Artificial
  Synthetic primer
tgacaagaccggcgccggtagcggcagtggtagtggta

SEQ ID NO: 10 - DNA, 38 bp, Artificial
  Synthetic primer
ggcctcgagcggcgccggggatccttaatgatggtgat

SEQ ID NO: 11 - DNA, 57 bp, Artificial
  Synthetic primer
cgacggcgccggatcctggagccacccgcaatttgaaaaagaaaacctgtattttca

SEQ ID NO: 12 - DNA, 57 bp, Artificial
  Synthetic primer
tgaaaatacaggttttctttttcaaattgcgggtggctccaggatccggcgccgtcg